(19) United States
(12) Patent Application Publication
(10) Pub. No.: US 2021/0213264 A1
(43) Pub. Date: Jul. 15, 2021

(54) MICRONEEDLE SYSTEM AND METHOD OF FABRICATION OF AN INGESTIBLE STRUCTURE

(71) Applicant: UNIVERSITY OF MARYLAND, COLLEGE PARK, COLLEGE PARK, MD (US)

(72) Inventors: SANWEI LIU, SAN JOSE, CA (US); SANGWOOK CHU, SAN JOSE, CA (US); REZA GHODSSI, POTOMAC, MD (US); LUKE BEARDSLEE, ATLANTA, GA (US); ASHLEY AUGUSTINY CHAPIN, WASHINGTON, DC (US)

(21) Appl. No.: 17/149,277

(22) Filed: Jan. 14, 2021

Related U.S. Application Data
(60) Provisional application No. 62/961,062, filed on Jan. 14, 2020.

(51) Int. Cl. A61M 37/00; A61L 31/10; B33Y 10/00; B33Y 80/00; B29C 64/135; B29C 64/30

(52) U.S. Cl. CPC A61M 37/0015; A61L 31/10; B33Y 10/00; B33Y 80/00; B29L 2031/7546; B29C 64/30; A61M 2037/0053; A61M 2205/0238; A61M 2210/1042; B29C 64/135

(57) ABSTRACT

A microneedle system includes an ingestible structure and one or more microneedle units secured to a surface of the structure. Each microneedle unit has a plurality of barb members extending from the outer surface of a microneedle. The microneedle unit is secured to the structure by a displacement member, which displaces the microneedle unit when in a released state. The displacement member may be held in a compressed state by a dissolvable coating.

### RELATED PATENTS AND APPLICATIONS

This application is based on U.S. Provisional Patent Application No. 62/961,062, filed on Jan. 14, 2020, which is incorporated herein by reference.

### STATEMENT REGARDING FEDERAL SPONSORED RESEARCH OR DEVELOPMENT

This invention was made with US Government support under Award No. ECCS1738211, awarded by the National Science Foundation. The US Government has certain rights in the invention.

### BACKGROUND OF THE INVENTION

The subject system and method are generally directed to a microneedle system providing releasable attachment of an ingestible structure to internal tissues of a subject's body. The system and method provide an ingestible structure having microneedles which are capable of anchoring to gastrointestinal tissue.

Gastrointestinal (GI) tract disorders account for 100 million doctors' visits a year. There is a significant need for minimally invasive technology to enable GI tract-targeted research, prescreening, diagnosis, and therapy, ultimately aiming towards advanced precision healthcare. Ingestion is a convenient and minimally invasive means to place medical devices within the GI tract. Therefore, ingestible capsules and other structures have demonstrated many acute applications while autonomously traversing the GI tract of a patient or other subject. However, the digestive process keeps such structures moving through the GI tract, placing a limit on how long they will remain in the subject's body. A means for holding such a structure within the GI tract for a prolonged period, which does not involve the invasiveness of surgical implantation, is therefore desirable.

### SUMMARY OF THE INVENTION

It is an object of the disclosed system and method to provide a system for non-invasive long-term attachment of a structure to internal tissues of a gastrointestinal tract, in order to achieve stationary positioning of the structure therein.

It is another object of the disclosed system and method to achieve a high pull-out/penetration ratio for the anchoring of the ingested structure to the internal tissues.

It is yet another object of the disclosed system and method that the system be simplified in its fabrication and operation.

It is a further object of the disclosed system and method to provide a releasable attachment of an ingestible structure to internal tissues of a subject's body which defines a structure body member with an external surface where a microneedle unit is secured to and displaceable from the surface of the ingestible structure.

It is a still further object of the disclosed system and method to provide a microneedle unit which is both secured to and displaceable from the surface of an ingestible structure with a displacement member secured on opposing ends to the microneedle unit and the external surface of the ingestible structure with the microneedle unit being displaceable from the ingestible structure into engagement with internal tissues of a subject's body.

These and other objects may be attained in a microneedle system and method of fabrication of an ingestible structure. In accordance with certain embodiments of the present invention, a system is provided for releasable attachment of an ingestible structure to internal tissues of a subject's body. The system includes an ingestible structure defining a structure body member having an external surface. The system also includes a microneedle unit secured to and displaceable from the surface of the ingestible structure. The system also includes a displacement member secured on opposing ends to the microneedle unit and the external surface of the ingestible structure. Said microneedle unit is adapted to be displaceable from the ingestible structure into engagement with the internal tissues of the subject's body.

In accordance with certain other embodiments of the present invention, a method is provided for fabricating a microneedle unit. The method includes providing a coverslip, and lithographically fabricating a microneedle on the coverslip. The microneedle has a tip, a base opposite the tip, an outer surface between the tip and the base, and a plurality of barb members secured to the outer surface and extending from the outer surface. The microneedle is fabricated tip-first with the tip in contact with a surface of the coverslip.

In accordance with certain other embodiments of the present invention, a method is provided for providing releasable tissue attachment functionality to an ingestible structure. The method includes providing a substrate. The method further includes lithographically fabricating a microneedle unit and a displacement member on a surface of the substrate. The displacement member is secured to the microneedle unit at a first end of the displacement member. The microneedle unit includes a microneedle defining a microneedle outer surface and a microneedle axis line extending in a longitudinal direction, and a plurality of barb members secured to the microneedle outer surface and extending from the microneedle outer surface. The plurality of barb members are adapted for engagement with internal tissues of a subject's body. The method further includes securing a second end of the displacement member opposite the first end to an external surface of a structure body member of said ingestible device. The method further includes applying a dissolvable coating to contain the microneedle unit and the displacement member and thereby place the displacement member in a compressed state.

Additional aspects, details, and advantages of the disclosed system and method are set forth, in the description and figures which follow.

### BRIEF DESCRIPTION OF THE DRAWINGS

FIG. 1A is a rendering of a proboscis of a spiny-headed worm (Acanthocephala);

FIG. 1B is an SEM (scanning electron microscope) image of a proboscis of a spiny-headed worm (Acanthocephala);

FIG. 2A is a schematic diagram illustrating an example of a microneedle unit, in accordance with an exemplary embodiment of the present invention;

FIG. 2B is a depiction of the microneedle unit of FIG. 2A in contact with an intestinal wall, in accordance with an exemplary embodiment of the present invention;

FIGS. 2C and 2D are SEM (scanning electron microscope) images of a microneedle unit, in accordance with an exemplary embodiment of the present invention;

FIG. 3 is a flow diagram illustrating a flow of processes for fabricating a microneedle unit, in accordance with an exemplary embodiment of the present invention;

FIGS. 4A-4E are schematic diagrams illustrating stages of the fabrication of the microneedle unit according to the processes of FIG. 3;

FIG. 5A is a schematic diagram illustrating an example of an ingestible structure, in accordance with an exemplary embodiment of the present invention;

FIG. 5B is a schematic diagram illustrating an example of an ingestible structure, in accordance with an alternate exemplary embodiment of the present invention;

FIG. 5C is a schematic diagram of a microneedle unit and displacement member secured to the surface of the ingestible structure of FIG. 5B, with the displacement member in a compressed state, in accordance with an exemplary embodiment of the present invention;

FIG. 5D is a schematic diagram of a microneedle unit and displacement member secured to the surface of the ingestible structure of FIG. 5B, with the displacement member in a released state, in accordance with an exemplary embodiment of the present invention;

FIG. 5E is an SEM image of a microneedle unit and displacement member, with the displacement member in a released state, in accordance with an exemplary embodiment of the present invention;

FIG. 6 is a flow diagram illustrating a flow of processes for providing releasable tissue attachment functionality to an ingestible structure, in accordance with an exemplary embodiment of the present invention;

FIGS. 7A-7H are schematic diagrams illustrating stages of the provision of the releasable tissue attachment functionality according to the processes of FIG. 6;

FIG. 8A is an SEM image of a microneedle tip penetrating tissue, with corresponding measurements, in accordance with an exemplary embodiment of the present invention;

FIG. 8B is an SEM image of a microneedle tip being extracted from tissue, with corresponding measurements, in accordance with an exemplary embodiment of the present invention;

FIGS. 8C and 8D are SEM images of the surface of a microneedle following penetration and extraction, in accordance with an exemplary embodiment of the present invention;

FIG. 9 depicts variations on an exemplary embodiment of a microneedle unit, with corresponding measurements, in accordance with the present invention; and

FIG. 10 is a chart of a typical compression and release force measurement profile of a displacement member, in accordance with an exemplary embodiment of the present invention.

### DETAILED DESCRIPTION OF THE PREFERRED EMBODIMENTS

Reference will now be made in detail to exemplary embodiments, which are illustrated in the accompanying drawings, wherein like reference numerals refer to the like elements throughout. The embodiments are described below in order to explain the disclosed system and method with reference to the figures illustratively shown in the drawings for certain exemplary embodiments for sample applications.

Section headings are included in this Detailed Description. It is noted that these section headings are for convenience only and should not be interpreted as limiting the scope of the claimed invention in any way.

The reduction in size of technological components has permitted the development of numerous ingestible devices for medical or other purposes. Generally, due to the fact that the digestive process of a subject's body moves ingested contents through the gastrointestinal (GI) tract in a predictable manner, such devices do not remain in any one place in the subject's body for extended periods of time.

A GI resident device is more specifically any device of an ingestible size which would be useful to maintain a position within the GI tract of a subject. One of skill in the art can imagine many devices and applications which would benefit from this function. Applications include but are not limited to a dispensing capsule for slowly releasing medication directly into the GI tract in a controlled fashion over an extended period of time; a sensor for monitoring of physiological conditions—e.g. acidity or bacteria levels—within the tract; or a probe for collecting a series of content samples from the tract.

One of the key limitations for GI resident device anchoring is a need for a robust tissue-anchoring mechanism that maintains resilience while immobilized in the wet, slippery mucosa during peristaltic contractions, while simultaneously minimizing tissue damage when being removed from the subject's body. While existing technologies have demonstrated mucosal anchoring strong enough to resist peristaltic movements, the preloading step (i.e. pressing down on mucosa surface) essential to current techniques, are challenging in that they require the integration of components that can apply the needed pressure for preloading in a compact format.

### 1. Spiny Microneedles

Studies of North American porcupine quills and worker honeybee stingers have revealed the importance of surface microbarbs for tissue-anchoring, yielding a structure that can achieve a larger pull-out force compared to the penetration force into skin and muscle tissues. These studies have provided useful insights into how to characterize and enhance tissue-anchoring strategies toward reducing the applied force (i.e., preloading).

The development of bio-inspired barbed microneedles has been limited by the need for high-resolution 3-D fabrication methods and the lack of materials able to successfully mimic the biological models. From the studies thus far, the tissue anchoring forces of barbed microneedles only allow for a pull-out/penetration ratio (PPR) of about 2. While the required PPR may exceed those demonstrated for previously developed biomimetic approaches, considering the target GI environment and size scale in the capsule context, such has motivated further investigation into elevated difference between the penetration and pull-out forces.

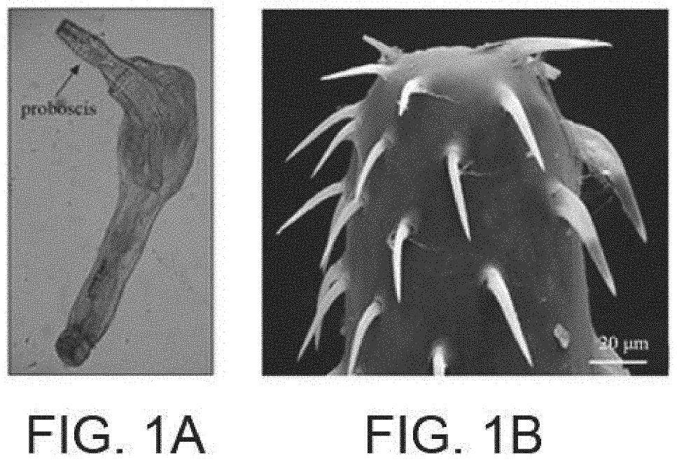

It has been determined that the spiny-headed worm (Acanthocephala), which lodges itself within the gut wall of its hosts with minimal locomotion, is believed to be a particularly promising biological model. The spiny-headed worm utilizes a proboscis to invaginate itself into the mucosa and backward-facing, sclerotized surface microhooks pierce the surrounding tissue, acting as mechanical anchors. An artist rendering and an SEM (scanning electron microscope) image of this proboscis are respectively provided in FIGS. 1A and 1B.

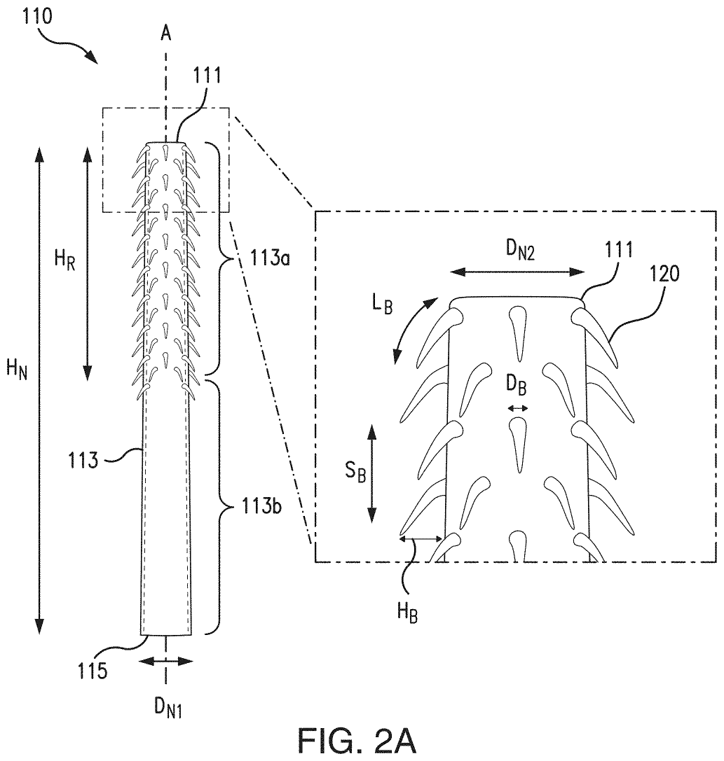

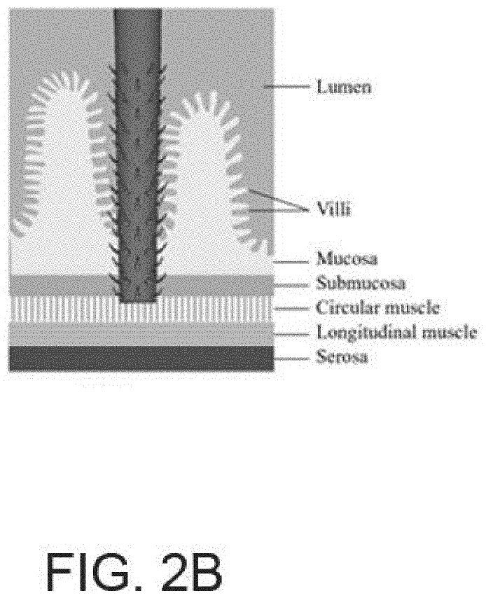

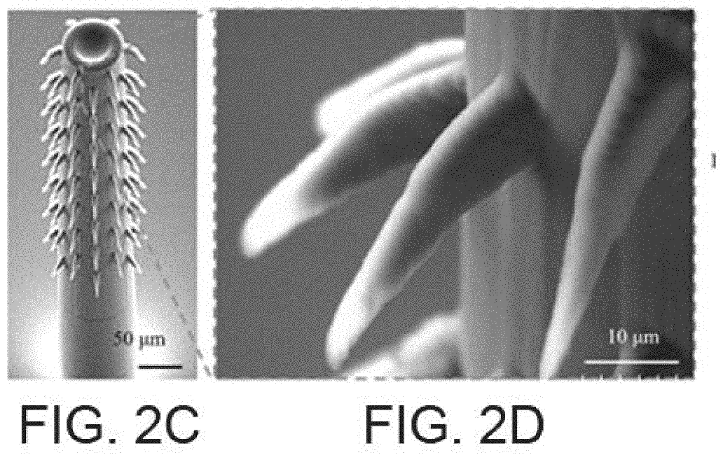

Based upon the concept inspired by the spiny-headed worm, Applicant has developed a microneedle unit, an embodiment of which is illustrated in FIG. 2A. The microneedle unit includes a microneedle 110, and a plurality of barb members or microhooks 120.

The microneedle 110 has a tip 111, a base 115, and an outer surface 113 therebetween. The microneedle 110 defines a microneedle axis line A extending in a longitudinal direction. The tip 111 and base 115 of the microneedle 110 may also be treated as the tip and base of the microneedle unit 10, and will be described as such throughout this disclosure.

The outer surface 113 includes a barbed region 113a. In the illustrated embodiment, the outer surface 113 also includes an unbarbed region 113b, although this is not a requirement, and in alternate embodiments the barbed region 113a covers all or substantially all of the outer surface 113. Also, in certain embodiments, multiple distinct barbed and unbarbed regions are present. In the illustrated embodiment, the barbed region 113a extends from the tip 111 of the microneedle for a predefined distance H_{R}, which therefore defines the height of the barbed region 113a.

The barb members 120 are distributed over and secured to the outer surface 113 of the microneedle 110, within the barbed region 113a. The barbed region 113a can therefore be termed a distribution portion of the outer surface 113. The barb members 120 are preferably arcuately contoured and directed radially with respect to the microneedle axis line A toward the base 115 of the microneedle 110. The barb members 120 are each adapted for engagement with internal tissues of a subject within the GI tract, in a manner which will be further elaborated upon.

The design of this microneedle unit is preferably tailored toward intestinal tissue morphology and fabrication feasibility. Without limitation, certain embodiments of the microneedle unit include one or more of the following features to better accomplish one or more of these objectives:

1) A height H_{N} and base diameter Dₘ of the microneedle 110 are similar in scale to human small intestinal villi (˜0.5 mm tall with ˜150 μm interstitial spacing), allowing optimal contact between the barb members 120 and an intestinal wall of the subject, as illustrated in FIG. 2B. Plainly, a greater H_{N} and/or smaller D_{N1} will also be effective for this purpose.

2) A height H_{N} of the microneedle 110 of ≥1 mm also allows the barb members 120 to penetrate the intestinal wall deeply enough to pass the mucus blanket and epithelial barrier.

3) A hollow microneedle 110 with a wall of ˜15 μm thickness saves over 50% of the laser writing time, and its increased flexibility potentially helps to avoid fracture while in tissue.

4) A blunt tip 111 of the microneedle 110 allows more barb members 120 to be evenly arranged around the tip 111, which has been determined to be the most critical portion for robust tissue anchoring, therefore maximizing the tissue anchoring pull-out force. A tip microneedle diameter D_{N2} of 74 μm will allow for six barb members 120 surrounding the tip 111.

5) The barb members 120 are distributed in a “quincuncial” pattern in the barbed region 113a of the outer surface 113, as illustrated in FIG. 2A. At a height H_{R} of 0.5 mm for the barbed region 113a, an axial spacing S_{B} of 60 μm between barb members 120, a base microneedle diameter D_{N1} of 150 μm, and a tip microneedle diameter D_{N2} of 74 μm, this will allow for ninety-six barb members 120.

6) The barb members 120 present a backward-facing curvature (preferably, ˜80°). A length L_{B} of the barb members 120 varies depending on the position on the surface 113 of the microneedle 110, decreasing for barb members 120 positioned further from the tip 111 (preferably, in an overall range of between 30 and 40 μm). The barb members 120 have a base diameter D_{B} of 8 μm, and a sharp tip to minimize the penetration resistance while maximizing tissue anchoring.

3-D TPP technology is utilized to achieve high fidelity fabrication with micrometer-scale resolution via an inversely oriented polymerization strategy. The excellent PPR achieved in this work provides opportunities to reduce the potential burdens of mechanical control and power consumption required for GI tissue anchoring, making the implantation of prolonged resident devices more feasible.

A fabricated version of the microneedle unit has been imaged by scanning electron microscope, and the images presented in FIGS. 2C and 2D.

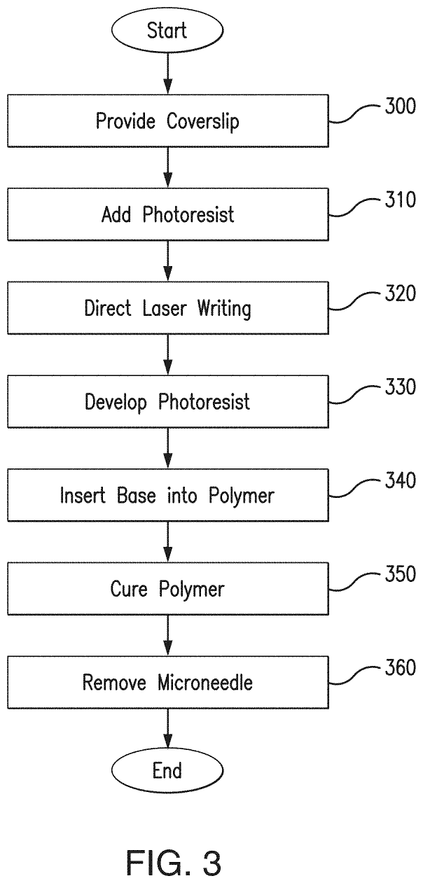

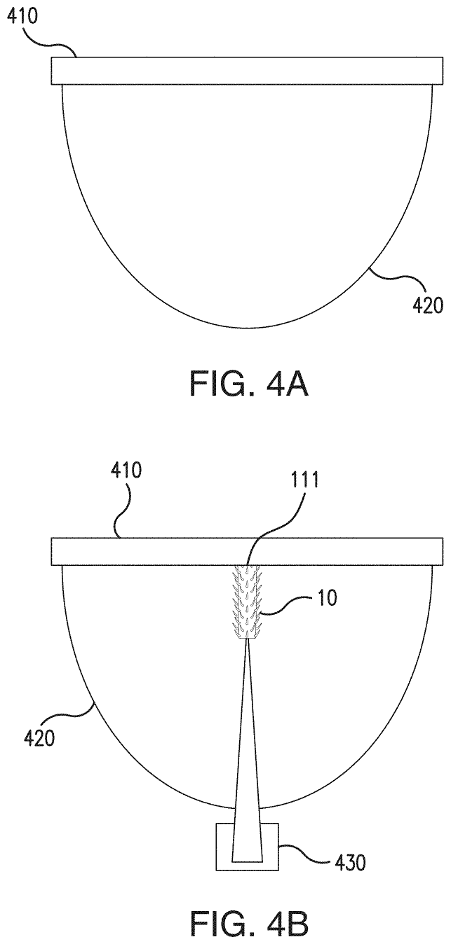

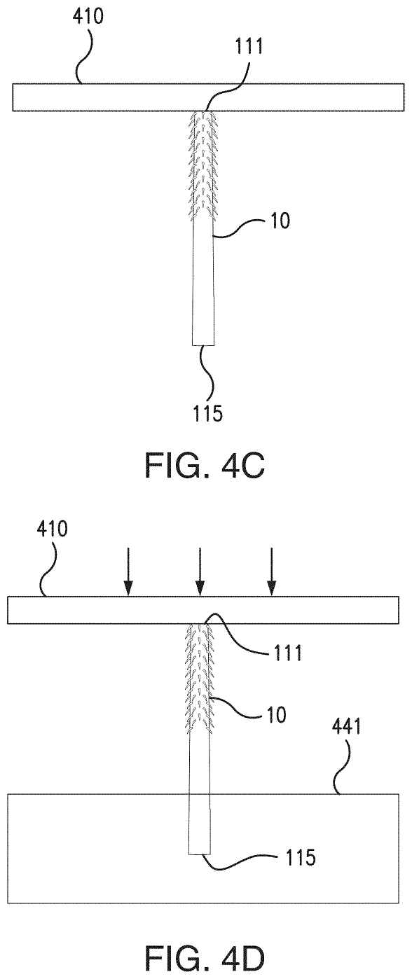

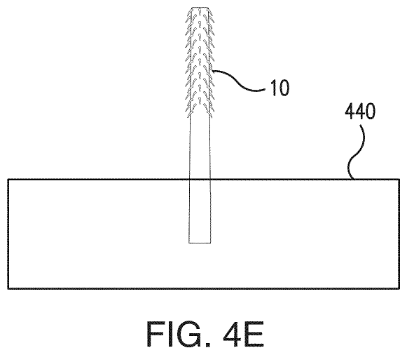

A method of fabricating a microneedle unit is illustrated in FIG. 3, with the fabrication stages illustrated in FIGS. 4A-4E, according to an embodiment of the invention. In the illustrated embodiment, the microneedle unit is constructed by a TPP lithography process with an inverse writing orientation (vs. scanning from the bottom), which is ideal for obtaining the precise geometric curvature (angle and sharpness) of the overhanging barb members 120 as it allows printing of a contiguous structure without free floating material.

At 300, a substrate or coverslip 410 is provided. In at least one embodiment, the substrate is made of glass. At 310, and as illustrated in FIG. 4A, a droplet 420 (preferably, ˜20 μL) of negative-tone photoresist is dispensed on the coverslip 410. At 320, and as illustrated in FIG. 4B, a tightly focused laser 430 initiates 3D polymerization of a microneedle unit 10, starting from the microneedle tip 111 (that is, tip-first). The microneedle 110 and barb members 120 are thereby lithographically fabricated on the coverslip 410 using direct laser writing. At 330, and as illustrated in FIG. 4C, the photoresist droplet 420 is developed and cleaned, leaving only the inversely-oriented microneedle unit 10, with the tip 111 of the microneedle 110 in contact with the coverslip 410.

At 340, and as illustrated in FIG. 4D, the base 115 of the microneedle 110 is inserted into a viscous polymer 441. At 350, the polymer is cured and cooled to form a solid substrate 440. Thereafter, the microneedle base 115 is firmly attached to the solid substrate 440, and the microneedle unit 10 can be easily peeled off of the coverslip 410 at 360, as illustrated in FIG. 4E. This leverages the weak adhesion between the coverslip 410 and the microneedle unit 10. The microneedle unit 10 is thereby transferred to the solid substrate 440. The polymer is preferably selected to form a flexible backing solid substrate, and can include polydimethylsiloxane (PDMS) or other suitable polymers.

It will be apparent that a plurality of microneedle units 10 can be generated on the same coverslip 410 in a desired arrangement, and simultaneously inserted into contiguous viscous polymer 441 which will be cured into a single substrate 440. The plurality of microneedle units 10 will then maintain the arrangement on the substrate 440.

The substrate 440 can be subsequently attached to, or utilized as, the surface of a larger structure, using any suitable means known in the art. Alternatively, the microneedle unit 10 can be detached from the substrate 440 by any suitable means known in the art.

In one example implementation of the fabrication method, an SMN 3D model is created using computer-aided design (CAD) software, such as SolidWorks (Dassault System, France), in the format of stereolithography (STL). The CAD file is then imported into computer-aided manufacturing software, such as DeScribe (Nanoscribe GmbH, Germany), for fabrication coding. Using, for example, Photonic Professional GT system (Nanoscribe GmbH, Karlsruhe, Germany), the design is fabricated via the Dip-in Laser Lithography (DiLL) objective (25× magnification, NA=0.8) with negative-tone photoresist (such as IP-S, Nanoscribe or Ormocomp, MicroChem) at 80 mW or 60 mW laser power and 40 mm/s scan speed (galvo mode), to achieve 1 μm layer height (150 μm splitting, piezo mode for z-axis), 500 nm hatching distance, and 8 minute processing time. After the laser writing, the sample is immersed in propylene glycol monomethyl ether acetate (PGMEA) to develop for 20 minutes, followed by a 2 minute isopropyl alcohol (IPA) cleaning. After the fabrication, the needle is assembled via dipping the needle base into 10:1 PDMS (Sylgard 184, Dow Corning, Corning, N.Y.) to form a 1 mm tall soft backing (0.25 mm penetration with a 4 minute curation time at 100° C.). Of course, the invention is not limited to this specific implementation, and numerous suitable substitutions at any stage can be imagined by those of skill in the art.

### 2. Ingestible Structure Employing Microneedle Units

A plurality of microneedle units on a surface of an ingestible structure will engage with internal tissues of a subject's body, within the gastrointestinal (GI) tract, after the subject has swallowed the ingestible structure. The ingestible structure will then maintain position against the internal tissues until removed by sufficient force. Due to the noted advantages of the disclosed microneedle units, the required force exceeds that of natural bodily functions, at least initially.

Removal of the microneedle units, and the ingestible structure, can be still achieved actively by applying a strong enough force to overcome the force applied by the barbs, via integration of suitable detachment mechanisms known in the art, such as a microactuator and magnetic modulation.

Removal can also be achieved passively over the aging process of the microneedles, wherein natural forces may overcome attachment forces over an extended period of wear and tear. Suitable means for aging the microneedles at a desired rate, such as the use of materials which will slowly dissolve under the conditions of the GI tract, and/or the deliberate introduction of defects into the needles or barbs which will cause them to break over time, are known in the art and will not be described further herein.

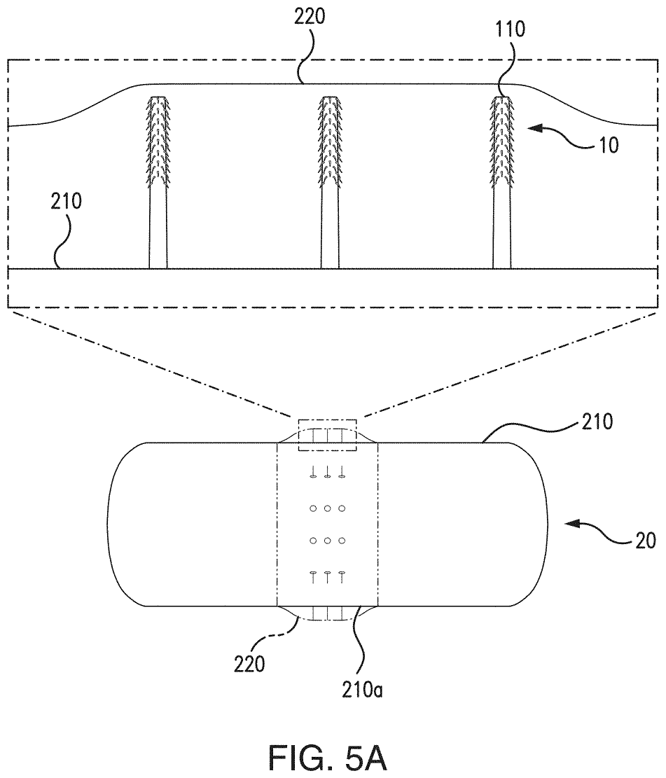

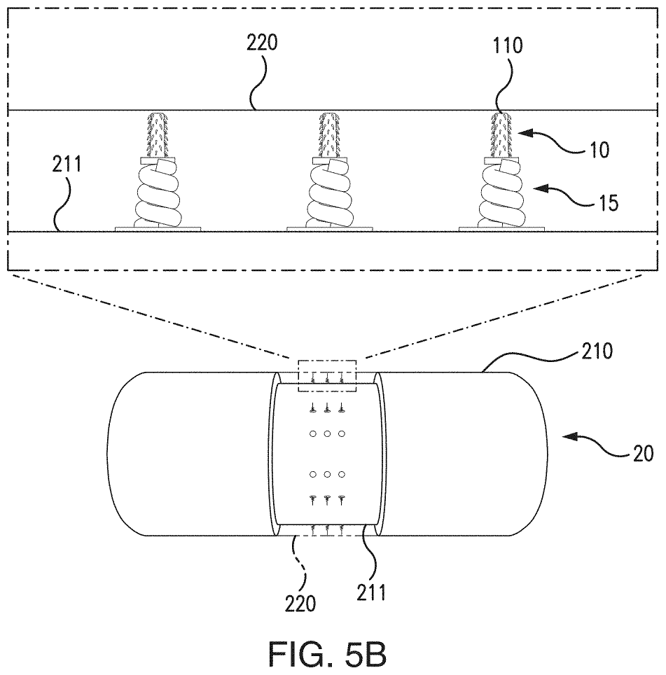

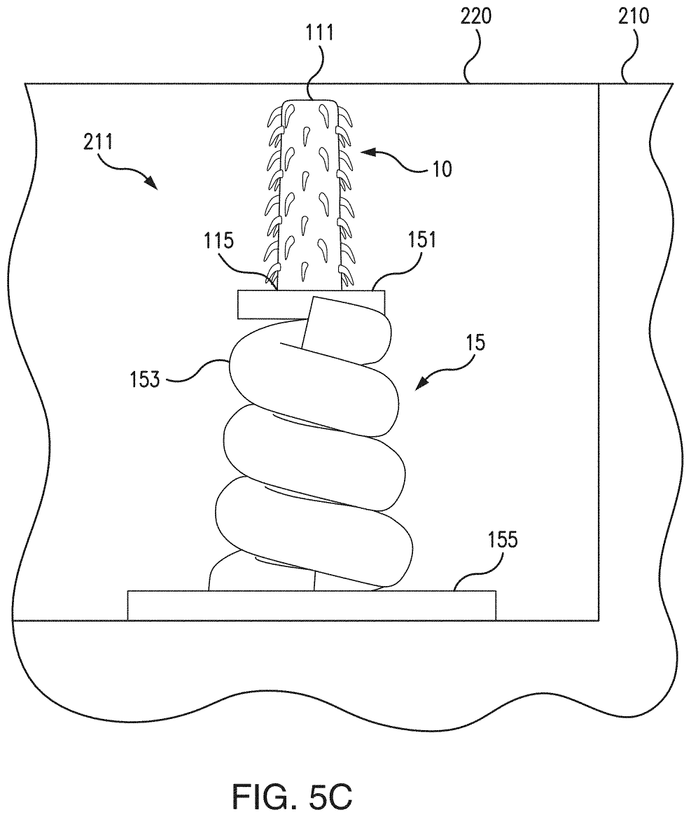

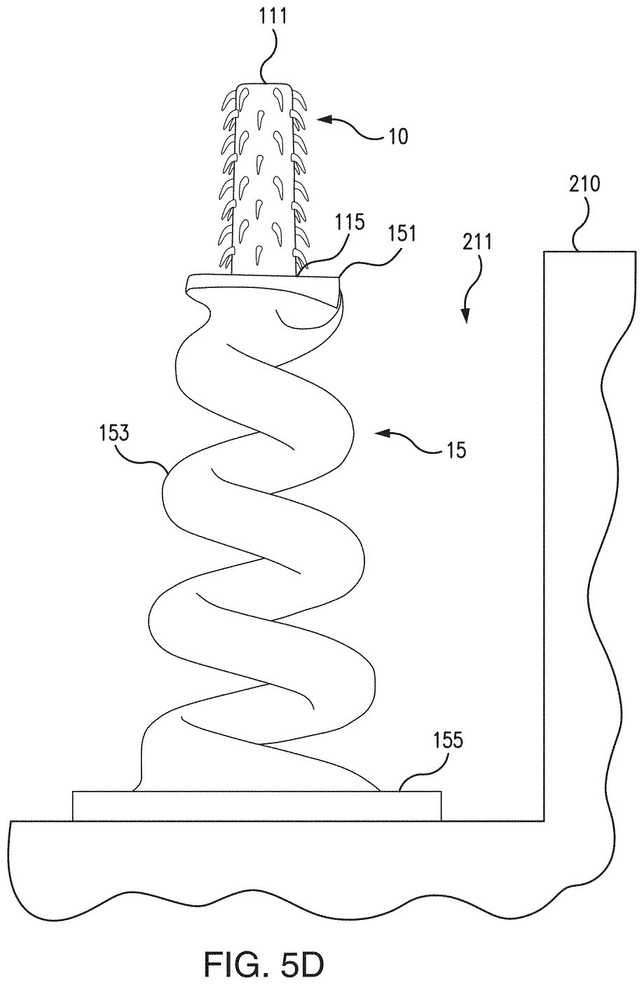

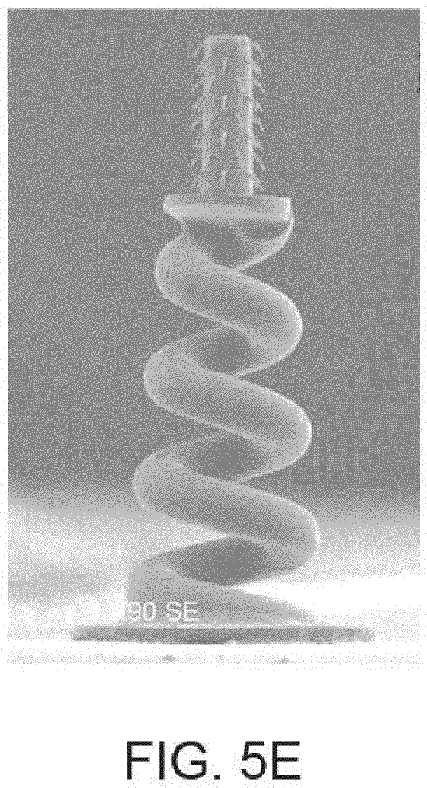

FIG. 5A is an illustration of an ingestible structure with microneedle units affixed thereto, according to an embodiment of the invention.

The purpose of the ingestible structure 20 is not limited by the present invention, and numerous applications are possible and have been previously listed herein. As such ingestible structures are known in the art, most elements of the structure 20 will not be described or depicted in detail, as they will vary depending on the particularly intended application.

The ingestible structure 20 has an external surface 210. At least one microneedle unit 10 is secured to the external surface 210. Preferably, and as illustrated in FIG. 5A, a plurality of microneedle units 10 are included and distributed so as to direct their tips 111 in a plurality of directions, so as to provide as many possible points and angles of engagement with internal tissues as reasonably possible. It is noted that the depicted number and arrangement of microneedle units 10, and their scale relative to the structure 20, are not limiting, but have been selected purely for convenience of depiction.

The external surface 210, or a needle portion 210a on which the microneedle units 10 are distributed, is in certain embodiments made at least partially of a solid substrate 440 fabricated as described in FIG. 3.

It is of course not desirable for the ingestible structure 20 to attach to tissues before the subject has even swallowed the structure. Therefore, preferably, a dissolvable coating 220 is provided to cover and contain the microneedle units 10. The dissolvable coating 220 is made of any suitable substance which is at least semisolid in normal environmental conditions, but will dissolve in the conditions found in the GI tract. In certain embodiments, this dissolution is due to the increase in temperature melting the substance, or the work of stomach acids or other liquids in the tract. This dissolution therefore gradually exposes the microneedle units 10 after ingestion, such that they can engage with the internal tissues. The dissolvable coating 220 need only cover a region of the external surface 210 on which the microneedle units 10 are disposed, and in certain embodiments only immediately cover each such microneedle units 10. However, it will be recognized that covering other elements on the external surface 210 will be advantageous for certain ingestible structures 20, to protect these elements or allow for easier ingestion of the structure as a whole.

In at least one embodiment, the dissolvable coating 220 includes polyethylene glycol (PEG). The selection of PEG is based on its melting point (53-58° C.) and dissolution rate. The relatively low melting point allows melted PEG at 100° C. to have long enough transition time before solidification for assembly of the dissolvable coating 220. Additionally, PEG will gradually dissolve while in contact with a flow of 37-38° C. water, such as found in the GI tract, and is non-toxic. Furthermore, the dissolution time is suitably tailored to the needs of the particularly intended application by altering the molecular weight of the selected PEG: the higher the molecular weight, the slower the dissolution, and the further into the GI tract the ingestible structure 20 will travel before engaging the internal tissues. Other polymers can achieve a similar control by selecting for pH-specific dissolution, based on the pH expected in particular GI locations (e.g. low pH in the stomach, neutral pH in the small intestines).

FIG. 5B is an illustration of an ingestible structure with microneedle units affixed thereto, according to an alternative embodiment of the invention. FIG. 5C more specifically illustrates a microneedle unit affixed to the ingestible structure of FIG. 5B by use of a displacement member in a compressed state, and FIG. 5D illustrates the displacement member in a released state.

In the illustrated embodiment, the microneedle unit 10 is affixed to a displacement member 15. A top end 151 of the displacement member 15 is secured to the base 115 of the microneedle 110. A bottom end 155 of the displacement member 15 opposes the top end 151, and is secured to the external surface 210 of the ingestible structure 20. It is again noted that the depicted number, arrangement, and scale of the microneedle units 10 and displacement members 15 are not limiting.

The displacement member 15 includes an elastic member 153 or other displacement mechanism which provides reversible displacement of the microneedle unit as a whole. One preferred form of the elastic member 153 is a conical micro-spring, which provides reduced solid height for a compact design as each active coil is partially recessed within the next larger coil. The conical design also provides lateral actuation stability as the base coils have larger diameters (250 μm to 100 μm) with less tendency to buckle than conventional compression springs. However, other suitable springs or displacement mechanisms are within the scope of the invention.

In the illustrated embodiment, the microneedle units 10 and displacement members 15 are disposed in a recess 211 defined on the external surface 210 of the ingestible structure 20. This recess 211 is of a depth such that, when a displacement member 15 (or, more precisely, its elastic member 153) is in a compressed state, the tip 111 of the corresponding microneedle 110 is completely within the recess 211, as illustrated in FIG. 5C. However, when the displacement member 15 is in a released state, the tip 111 of the corresponding microneedle 110 projects outside the recess 211, as illustrated in FIG. 5D. As such, the microneedle unit 10 can only engage the internal tissues effectively while the displacement member 15 is in the released state. Preferably, the microneedle 110 is completely outside the recess 211 when the displacement member 15 is in the released state. The act of the release can further assist the microneedle 110 in penetrating internal tissues.

In the illustrated embodiment of FIGS. 5B and 5C, the displacement members 15 are maintained in the compressed state by the dissolvable coating 220, which completely or partially fills the recess 211. Once the dissolvable coating 220 has dissolved, as illustrated in FIG. 5D, the displacement members 15 all elastically extend to their released state and project the microneedle units 10 to engage the internal tissues.

However, other suitable means for compressing and releasing the elastic member 153 of the displacement members 15 are within the scope of the invention, and include but are not limited to various mechanical release mechanisms known in the art, which can be remotely actuated, or released on expiration of a timer or other condition. Such mechanisms have the additional advantage of being easily reversible.

A fabricated version of the microneedle unit in combination with the displacement member has been imaged by scanning electron microscope, and the image presented in FIG. 5E.

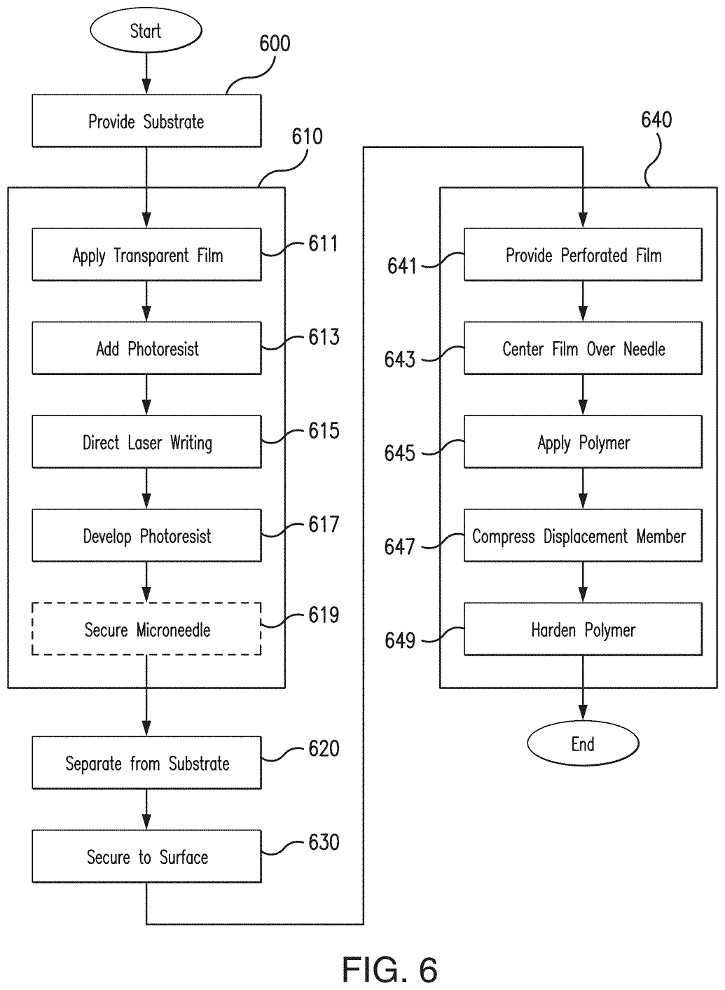

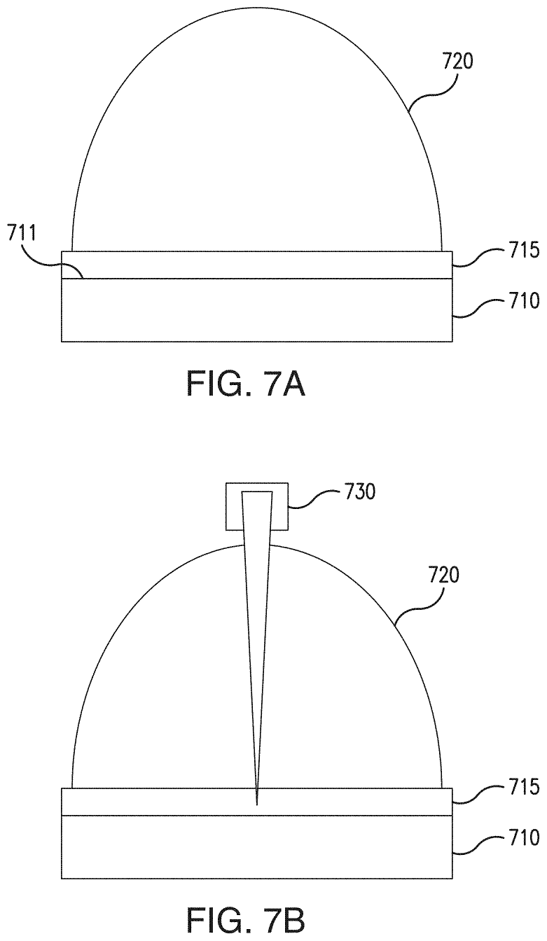

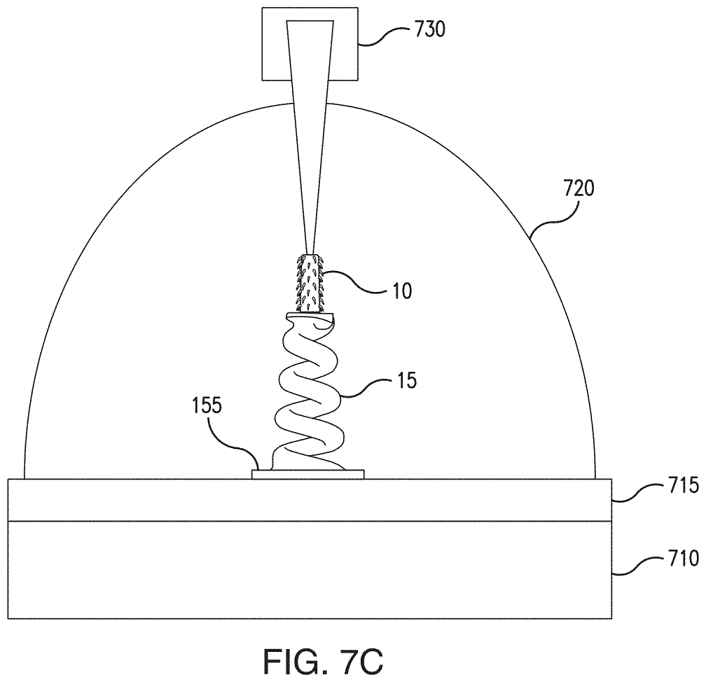

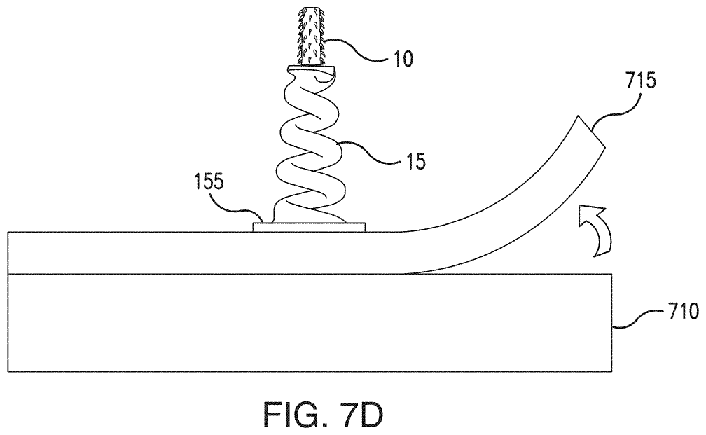

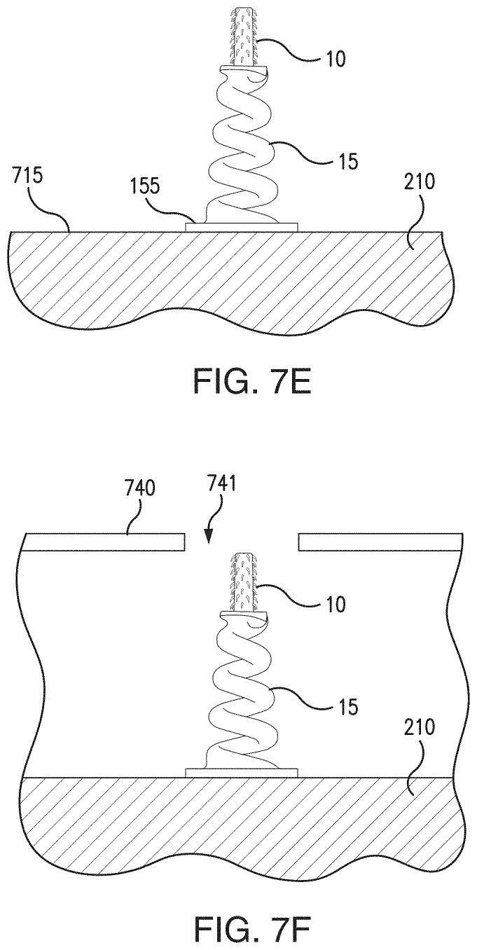

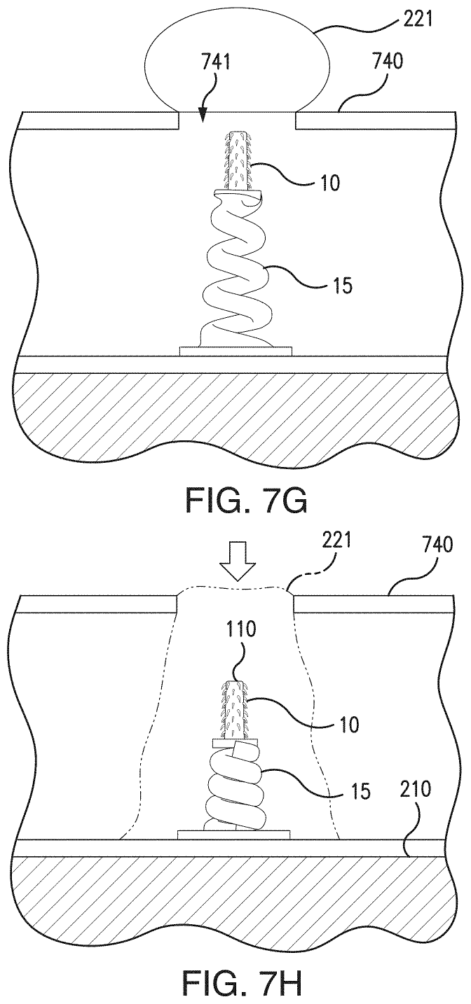

A method of providing releasable tissue attachment functionality to an ingestible structure 20 is illustrated in FIG. 6, with the fabrication stages illustrated in FIGS. 7A-7H, according to an embodiment of the invention.

At 600, a substrate or coverslip 710 is provided. In at least one embodiment, the substrate is made of glass, and is coated with indium tin oxide (ITO).

At 610, a displacement member 15 and microneedle unit 10 are fabricated on a surface 711 of the substrate 710. In a preferred embodiment, these components are fabricated as follows:

Preferably, at 611, a transparent film 715, such as a flexible polyimide substrate (Kapton tape), is applied to cover the surface 711 of the substrate 710. The transparency of the film allows the system to automatically locate the refractive interface on the surface of the substrate 710.

At 613, and as illustrated in FIG. 7A, a droplet 720 of negative-tone photoresist is dispensed on the substrate 710. At 615, and as illustrated in FIGS. 7B and 7C, a tightly focused laser 730 initiates 3D polymerization of the displacement member 15. The displacement member 15 is thereby lithographically fabricated on the substrate 710 using direct laser writing. If the transparent film 715 has been applied, the initial laser interaction is confined within the transparent film 715 and does not cure the photoresist, as illustrated in FIG. 7B, correspondingly reducing the thickness of a base portion at the bottom end 155 of the displacement member 15. At 617, the photoresist droplet 720 is developed and cleaned, leaving the results of the laser writing exposed on the substrate.

It is noted that, unlike in the embodiment illustrated in FIGS. 3 and 4A-4E, the fabrication illustrated here is bottom-up, starting with the bottom end 155 of the displacement member 15. In certain embodiments, the laser writing ceases at the top end 151 of the displacement member 15. Then, at 619, a microneedle unit 10 which has been separately fabricated, for example according to the embodiment illustrated in FIGS. 3 and 4A-4E or similar processes, is secured to the top end 151 using any suitable process known in the art.

However, in alternate embodiments, a single laser writing process fabricates both the displacement member 15 and the microneedle unit 10 as integrally formed with each other at 615. In this embodiment, the barb members 120 are initially created without any support due to the bottom-up fabrication, and are integrated with the microneedle 110 as the laser writing continues upward, as illustrated in FIG. 7C. One-step bottom-up fabrication of both displacement member 15 and the microneedle unit 10 reduces the effort of assembly of these two parts, with the slight disadvantage of obtaining lower curvature of the barb members 120 to allow them to be promptly attached to the microneedle 110 during fabrication, before drift can occur. In penetration/pull-out testing, there was minimal difference in performance between top-down and bottom-up fabricated microneedle units 10.

Once the fabrication is complete, at 620, the bottom end 155 of the displacement member 15 is separated from the substrate 710. Then, at 630, the bottom end 155 of the displacement member 15 is secured to the external surface 210 of the ingestible structure 20. If transparent film 715 has been applied to the substrate 710, this film provides an easy tool for such separation and transfer, as illustrated in FIGS. 7D and 7E.

At 640, the dissolvable coating 220 is applied to compress the displacement member 15 and contain the microneedle unit 10. More specifically, in the illustrated embodiment, at 641 a perforated film 740 is provided that has at least one hole 741 therein. At 643, and as illustrated in FIG. 7F, the hole 741 is centered over the microneedle unit 10. At 645, and as illustrated in FIG. 7G, a droplet 221 of polymer in an aqueous state is applied to the hole 741 of the film 740. For example, in certain embodiments, the polymer is in a melted or semi-melted condition. At 647, and as illustrated in FIG. 7H, the tip 111 of the microneedle 110 is pressed against the droplet 221, thereby compressing the displacement member 15, and at 649, the droplet is allowed to harden to form at least a portion of the dissolvable coating 220.

Preferably, the perforated film 740 has a pattern of holes 741 which are distributed to correspond to the positions of a plurality of microneedle units 10. A plurality of droplets 221 can then be applied and the plurality of microneedle units 10 can be contained simultaneously. However, it is within the scope of the invention that a perforated film 740 with a single hole 741 is used repeatedly, once for each microneedle unit 10.

One example embodiment will now be described in detail. In this example embodiment, the recess 211 has a depth of 750 μm. The microneedles 110 each have a height of 260 μm, with forty-eight barb members 120 distributed over the outer surface 113. The conical springs of each displacement member 15 contain four spring coils with an 80-μm wire diameter, with an overall base diameter of 250-μm, allowing stable directional actuation with an estimated spring constant of 340 N/m and 100 mN peak compression force at 300 μm displacement (as calculated based on the spring model, the measured Young's modulus, and the shear strength of IP-S photoresist). In a released state, each displacement member 15 has a height of 715 μm. Kapton tape on the substrate has a thickness of 25 μm, and a corresponding 25 μm of the bottom end 155 of the displacement member 15 is printed therein and does not cure, leaving a base thickness of 25 μm outside the Kapton tape.

In this example embodiment, the microneedle unit 10 and displacement member 15 are fabricated utilizing a high-precision DLW technology (Photonic Professional GT, Nanoscribe GmbH, Germany), which is based on highly localized interactions between a femtosecond laser and photosensitive material. First, the 3D model of the SMU is created via SolidWorks (Dassault System, France) computer-aided design (CAD) software. The CAD file, in the format of stereolithography (STL), is then imported and processed in the DeScribe software for fabrication coding. The design is then printed using the Dip-in Laser Lithography (DiLL) objective (25× magnification, NA=0.8) with a negative-tone IP-S photoresist (Nanoscribe GmbH Germany, suitable for mesoscale prototyping compared to other photoresist) on Kapton tape on ITO-coated glass. The coded file is loaded into the DLW software, Nanowrite, for fabrication under the DLW settings of 50 mW laser power and 100 mm/s scan speed (galvo mode) to achieve 1-μm lateral slicing layer height, 1-μm hatching distance, and 26-minute fabrication time. After the laser writing, the sample is immersed in propylene glycol monomethyl ether acetate (PGMEA) to develop for 5 minutes, followed by 2-min of isopropyl alcohol (IPA) cleaning. Due to the limitation of the 300-μm by 300-μm lateral laser working area, the microneedle unit 10 and displacement member 15 are split into five parts to complete, with 2-μm crosslinking stitching overlapping.

The perforated film 740 is a 2-mm by 2-mm, 750-μm thick PDMS film, patterned with a 1-mm biopsy punch to create central holes. The droplet 221 of polymer in an aqueous state is PEG which has been pre-melted at 100° C., and hardens by cooling over two minutes while the displacement member 15 is compressed beneath by 225 μm.

### 3. Testing Results

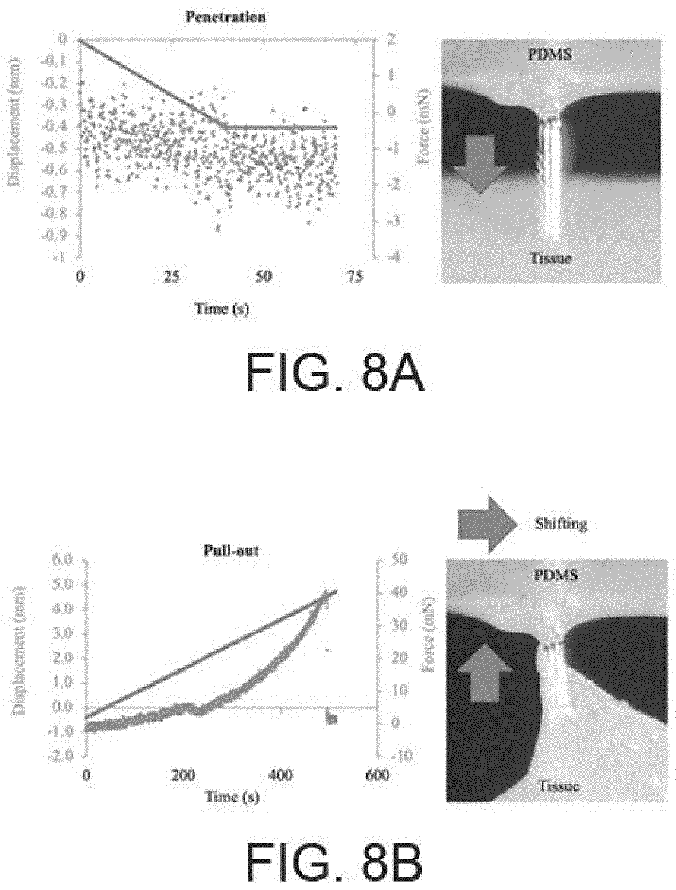

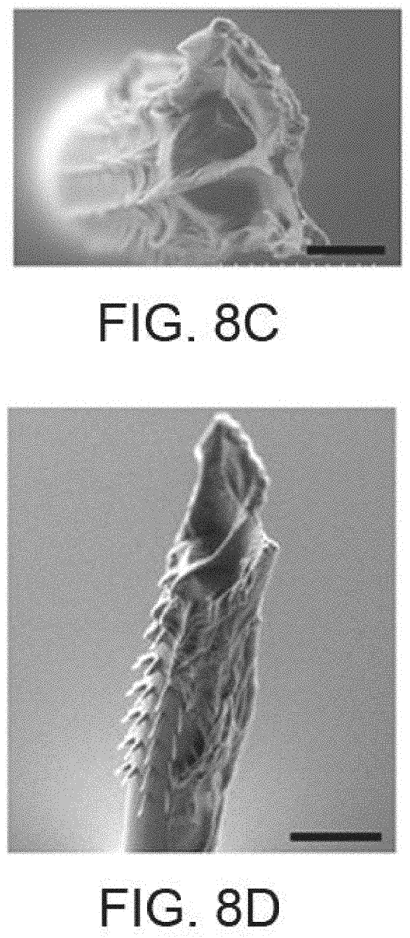

Displacements and forces during mechanical testing of tissue-anchoring were measured using the disclosed microneedle unit on segments of thawed porcine small intestinal tissue. Thawed porcine small intestinal tissue, purchased from Animal Biotech Industries Inc, PA, USA, is cut into an approximately 1 cm×1 cm patch and the non-mucosa side is attached with superglue to a 3-D printed fixture. The tissue sample is characterized using a universal testing machine (Model 5565, Instron, MA, USA) with tensile and compressive modes containing a +/−50 N load cell. A microneedle assembled on PDMS backing is attached to the movable sensing column. Upon balancing the load cell, the microneedle tip comes into contact with the tissue sample surface. During the test, the apparatus pushes the microneedle into the tissue mucosa at a rate of 0.01 mm/s until the displacement reaches 0.4 mm and holds the penetration for 30 s. This penetration is illustrated in FIG. 8A, with the displacements and forces illustrated in the corresponding chart.

After a 1 mm lateral shifting perpendicular to the penetration direction (mimicking the peristaltic shear interaction in the GI tract), the microneedle is then moved upward (away from the tissue sample) at a rate of 0.01 mm s-1 until the microneedle is pulled out of the tissue completely. This extraction is illustrated in FIG. 8B, with the displacements and forces illustrated in the corresponding chart.

As can be seen, compared to the penetration force of about −2 mN at 0.4 mm displacement, the 40 mN maximum pull-out force is about 20-fold larger than the penetration force. The average penetration and pull-out forces during this sequence are measured as −0.6 mN and 25 mN, respectively, with considerably large standard deviations (0.7 mN and 31 mN, respectively). The large standard deviation is partly due to system noise and more importantly, the number of barb members that become attached to the tissue sample. However, these factors are not critical as more than half of the samples demonstrated an over 20-fold PPR (P<0.05), suggesting significantly enhanced tissue anchoring performance.

The microneedle was imaged using a scanning electron microscope after testing. Images of the top view and side view are respectively presented in FIGS. 8C and 8D, with respective scale bars of 50 μm and 100 μm. These images display effective tissue attachment on the needle surface.

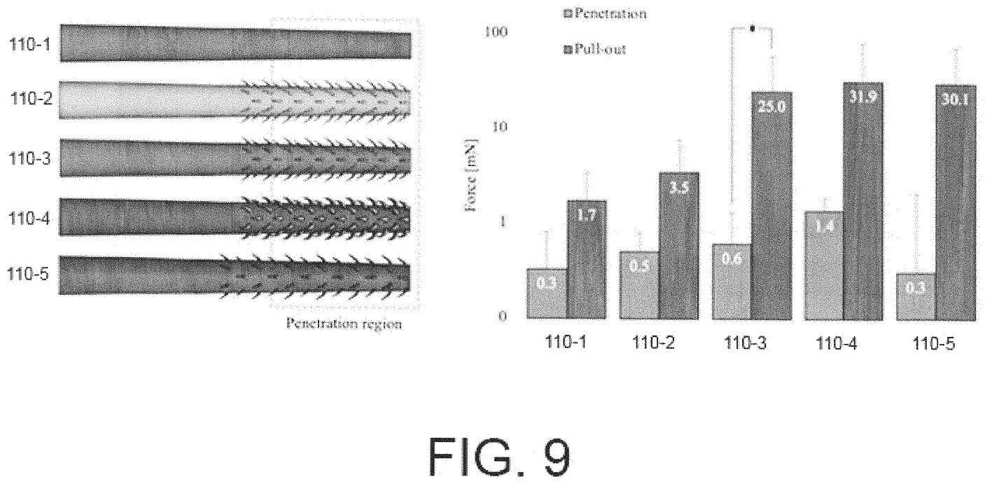

Further experimentation with the configuration of the barb members was performed to determine an ideal pull-out force to penetration force ratio (PPR). Schematics of these different configurations, and their corresponding effectiveness, are illustrated in FIG. 9.

As a control, microneedle 110-1 has no barb members and relies solely on the frictional force between the needle surface and the surrounding tissue in this anchoring process. As a result, there is no significant difference between the penetration and pull-out forces with both measuring low force levels.

Microneedle 110-2 is fabricated with a softer photoresist than microneedle 110-3, which uses IP-S, resulting in softer barb members (Young's modulus: 1 GPa vs. 4.6 GPa). Microneedle 110-2 provides less effective anchoring (>4-fold lower pull-out force) compared to the microneedle 110-3 with stiffer barb members. This indicates the importance of material stiffness in achieving robust mechanical interlocking between the barb members and the intestinal tissue. These finding may be affirmed by the evolutional success of the spiny headed worm's effective parasitic tissue anchoring structure (sclerotized surface hooks).

Microneedle 110-4 has an increased barb size of 16-μm base diameter, compared to microneedle 110-3 with 8-μm base diameter. This variation showed only a slight increase in pull-out force while the penetration force showed a 2-fold increase. This suggests that increasing the barb size brings marginal improvement to the strength of tissue anchoring while the increased contact area of the larger barbs leads to greater penetration resistance, ultimately resulting in a lower PPR.

Microneedle 110-5 reduces the number of barb members by increasing the axial spacing over microneedle 110-3, from 60-μm to 90-μm. Microneedle 110-5 demonstrated a tissue anchoring performance comparable to microneedle 110-3, with slightly decreased penetration force (0.3 mN), similar pull-out force (30 mN), and even higher PPR (˜97), suggesting a possibility for further development.

Overall, these tests determined that a smaller barb base diameter and lower density are beneficial for low penetration force, while microneedle stiffness increases the required pull-out force and thereby improves the tissue anchoring.

Compared to the magnetorheological drawing honeybee stingers which reported penetration force, pull-out force, and PPR of −42 mN, 73 mN, and ˜1.8 on rabbit skin, respectively, the microneedles of the present disclosure demonstrated a 1-2 orders of magnitudes lower penetration force with comparable pull-out force, resulting in a more than 10-fold enhancement in the PPR. Compared to those from natural honeybee stingers which reported penetration force, pull-out force, and PPR of −5.8 mN, 114 mN, and ˜20 on rabbit skin, respectively, the microneedles present even lower penetration force, lower pull-out force, and equivalent PPR. Considering the nearly 50% narrower diameter (108-μm vs. 200-μm) used in this research, these results imply a significant enhancement in tissue anchoring performance attributed to the advanced fabrication that closely replicates the parasitic tissue anchoring biological model.

Testing was also performed upon the preferred embodiment of the displacement member previously identified. A compression and release test showed that the conical spring can withstand a 300-μm compression displacement and fully recover after release. There was no spring coil damage observed during the test. Also, the displacement member remained adhered to the polyimide substrate during the test. The results indicate that the design characteristics are appropriate for the microneedle actuation.

The system was also quantitively tested using a universal testing machine (Model 5565, Instron, MA, USA) with tensile and compressive modes containing a +/−50 N load cell and 3D-printed fixture assembled to the upper sensing column to compress the samples. During the test, a microneedle unit was placed under the movable sensing column of the tester. After balancing the load cell, the upper movable column moved towards and pushed the microneedle unit with its fixture at the speed of 0.05 mm/s until the compression displacement of the displacement member reached 300 μm. After the compression, the upper column moved upwards to release the displacement member at the same speed. Throughout the test, the upper sensing column recorded time, displacement, and force measurement. This process was video recorded using a stereomicroscope (Leica M125, Germany) and a digital camera (Sony a6000, Japan).

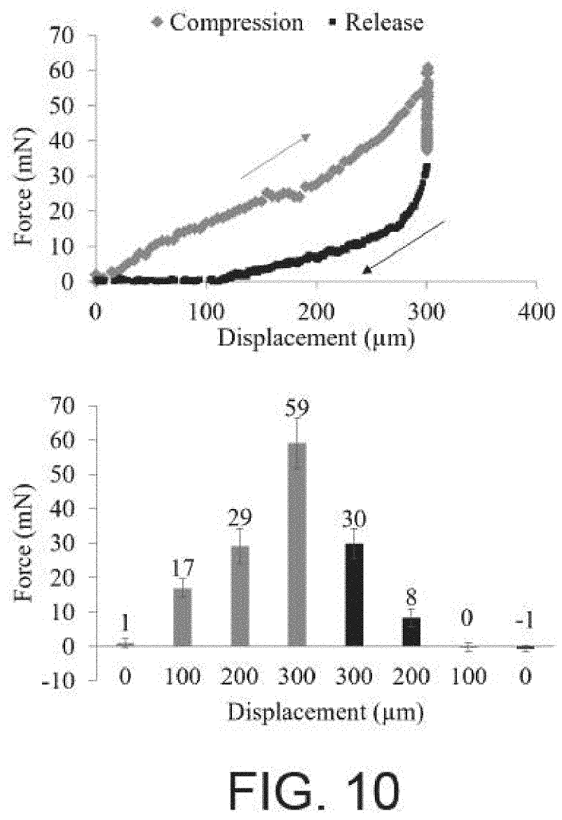

A resulting typical compression and release force measurement profile of the displacement member is illustrated in FIG. 10, and shows that the compressional force increases as the displacement increases. Before releasing, the compression force decreases from 59.3 mN to 32.7 mN, indicating stress relaxation. The difference between the compression and release (hysteresis) indicates that the SMU spring has a considerably large energy dissipation, possibly due to the internal friction of the building block material (IP-S, Nanoscribe Gmbh, Germany). The hold time of the compression of the displacement members are about 30s. Future work needs to address the total relaxation of the SMU to demonstrate long-term actuation performance. The average compression force increases up to 59 mN over a 300-μm displacement, showing an average spring constant of 197 mN/mm. The lower calibration values compared to the analytical estimations are attributed to the variation in the mechanical property of the cured photoresist (IP-S, Nanoscribe Gmbh, Germany). The releasing force, measured after stress relaxation, demonstrates 8 mN at 100-μm microneedle actuation relative to the maximally compressed state (equivalent to a 200-μm displacement from its position before loading), ultimately indicating sufficient mechanical rigidity to withstand the 1.6 mN microneedle penetration force and the 8 μN peristaltic force.

Additionally, cyclical mechanical tests of both single microneedle unit and 1×3 microneedle unit array are presented in the tables below:

REPRESESNTATIVE SINGLE SMU CYCLIC PERFORMANCE

| | Force at 300 μm | Force at 200 μm displacement |
|---|---|---|
| Cycle | displacement (mN) | or 100 μm actuation (mN) |
| Cycle 1 | 67 | 8 |
| Cycle 2 | 42 | 4 |
| Cycle 3 | 32 | 3 |
| Cycle 4 | 29 | 2 |
| Cycle 5 | 28 | 3 |

REPRESENTATIVE 1 ×
3 SMU ARRAY CYCLIC PERFORMANCE

| | Force at 300 μm | Force at 200 μm displacement |
|---|---|---|
| Cycle | displacement (mN) | or 100 μm actuation (mN) |
| Cycle 1 | 132 | 40 |
| Cycle 2 | 126 | 37 |
| Cycle 3 | 121 | 31 |
| Cycle 4 | 116 | 30 |
| Cycle 5 | 132 | 40 |

The measured recovery force at 200 μm displacement or 100 μm actuation demonstrated a large enough actuation force for robust tissue anchoring (>2 mN per microneedle). Notably, the 1×3 microneedle unit array presents a much higher actuation force, possibly due to secondary parasitic motions of individual microneedle units (e.g. lateral collapse) that provide interactive supports to each other, demonstrating the advantage of a microneedle unit array for future capsule integration

Testing was also performed upon a polyethylene glycol (PEG) embodiment of the dissolvable coating. Initially, a microneedle unit and displacement member are embedded in PEG after assembly, such as described with regard to FIGS. 6 and 7A-7G. As body temperature (38° C.) water droplets (˜0.06 mL) are gradually added onto the assembly, the microneedle tip slowly appears with a measured height from the base (including the substrate, displacement member, and microneedle unit) of 1.47 mm (h1) and 1.48 mm (h2) at t=13 min and t=16 min, respectively. At t=25 min, the entire microneedle unit appears with a measured height of 1.56 mm (h3). At t=72 min, the displacement member has also appeared, although with some PEG trapped within the space of the spring, with a measured height of 1.61 mm (h4). At t=103 min, the majority of the displacement member appears and the final height remains at 1.61 mm (h5). Because the initial displacement at t=0 cannot be directly measured (the microneedle not being visible when fully embedded in the PEG), the estimated minimum displacement recovery is 140 μm, determined via subtracting the displacement between h5 and h1. This measured actuation displacement is large enough for the microneedle to interact with GI tissue, confirming the autonomous actuation capability.

### 4. Conclusion

Overall, the bio-inspired design and fabrication of tissue anchoring microneedles demonstrate excellent mechanical characteristics. The 3-D printed spiny microarchitectures achieved exceptionally low tissue penetration forces yet maintained high pull-out forces, demonstrating an over 10-fold enhancement in pull-out/penetration performance compared to the state-of-the-art. The spiny barb members on the needle surface successfully performed as tissue interlocking anchors. The results suggest that appropriate mechanical stiffness plays an essential role to better tissue anchoring (resisting pull-out), while surface barb dimension and pattern density mostly contribute to tissue penetration. The advantages of the system over the art can be summarized as:

1. Use of micrometer resolution 3-D direct laser writing produces high fidelity biomimicry fabrication.

2. Barbed microneedles have penetration force lower than 1 mN and over 20-fold larger pull-out force, demonstrating easy penetration yet strong removal on GI tissue for the first time.

3. Penetration force is one-to-two orders of magnitude lower than the existing art, and pull-out to penetration ratio is over 10-fold larger.

4. Characterization of barb size, pattern, and material properties presents strategies for tissue-anchoring optimization.

5. Ease of tissue penetration and robust anchoring strength provide great opportunities for conveniently implanting devices embedded within the intestinal epithelium.

6. Use of this technology will ultimately support the development of ingestible capsule systems, particularly for deploying resident devices for extended monitoring and drug delivery in the GI tract.

Overall, the unprecedented tissue anchoring characteristics confer exciting opportunities for anchoring next-generation GI-resident devices, enabling personalized healthcare applications.

The descriptions above are intended to illustrate possible implementations of the disclosed system and method, and are not restrictive. While this disclosure has been made in connection with specific forms and embodiments thereof, it will be appreciated that various modifications other than those discussed above may be resorted to without departing from the spirit or scope of the disclosed system and method. Such variations, modifications, and alternatives will become apparent to the skilled artisan upon a review of the disclosure. For example, functionally equivalent elements or method operations are substitutable for those specifically shown and described, and certain features are usable independently of other features. Additionally, in various embodiments, all or some of the above embodiments are selectively combined with each other, and particular locations of elements or sequence of method operations are reversed or interposed, all without departing from the spirit or scope of the disclosed system and method as defined in the appended claims. The scope should therefore be determined with reference to the description above and the appended claims, along with their full range of equivalents.

What is claimed is:

1. A microneedle system for releasable attachment of an ingestible structure to internal tissues of a subject's body, comprising:
an ingestible structure defining a structure body member having an external surface;
a microneedle unit secured to and displaceable from said surface of said ingestible structure; and,
a displacement member secured on opposing ends to said microneedle unit and said external surface of said ingestible structure,
whereby said microneedle unit is adapted to be displaceable from said ingestible structure into engagement with said internal tissues of said subject's body.

2. The microneedle system as recited in claim 1, wherein said microneedle unit includes:
a microneedle defining a microneedle outer surface and a microneedle axis line extending in a longitudinal direction; and
a plurality of barb members secured to said microneedle outer surface and extending from said microneedle outer surface, said plurality of barb members adapted for engagement with said internal tissues of said subject's body subsequent to ingestion of said ingestible structure.

3. The microneedle system as recited in claim 2, wherein said barb members are arcuately contoured and directed radially with respect to said axis line of said microneedle toward said external surface of said ingestible capsule.

4. The microneedle system as recited in claim 2, wherein said barb members are distributed in a quincuncial pattern on said microneedle outer surface.

5. The microneedle system as recited in claim 2, wherein a spacing on said microneedle outer surface between each adjacent pair of said barb members is greater than or equal to 60 μm.

6. The microneedle system as recited in claim 2, wherein said barb members are disposed through a distribution portion of said microneedle outer surface defined between a tip of the microneedle and a predefined distance from the tip of the microneedle, the predefined distance being at least 0.5 mm.

7. The microneedle system as recited in claim 1, wherein said displacement member includes an elastic member for reversible displacement of said microneedle unit with respect to said external surface of said ingestible device.

8. The microneedle system as recited in claim 7, wherein said elastic member is a conical spring secured on opposing ends to said ingestible device and said microneedle unit.

9. The microneedle system as recited in claim 7, wherein said elastic member is a spring member maintained in a compressed state prior to ingestion of said ingestible structure.

10. The microneedle system as recited in claim 9, further comprising a dissolvable coating containing said microneedle unit and said compressed spring member prior to ingestion of said ingestible capsule, said dissolvable coating for dissolving subsequent to ingestion of said ingestible capsule to thereby release said spring member from said compressed state and displace said microneedle unit into engagement with said internal tissues of said subject's body.

11. The microneedle system as recited in claim 10, wherein a recess is defined on said external surface of said ingestible structure, said microneedle unit and said compressed spring member disposed in said recess, said dissolvable coating filling said recess.

12. The microneedle system as recited in claim 10, wherein the dissolvable coating includes polyethylene glycol.

13. A method for fabricating a microneedle unit, comprising:
providing a coverslip; and
lithographically fabricating a microneedle on said coverslip, said microneedle having a tip, a base opposite said tip, an outer surface between said tip and said base, and a plurality of barb members secured to said outer surface and extending from said outer surface, said microneedle being fabricated tip-first with said tip in contact with a surface of said coverslip.

14. The method as recited in claim 13, further comprising transferring said microneedle to a flexible backing substrate, said base of said microneedle thereby affixed to said flexible backing substrate.

15. The method as recited in claim 14, wherein said microneedle is transferred to said flexible backing substrate by:
inserting said base of said microneedle into a polymer in a viscous state;
curing said polymer to form said flexible backing substrate; and
separating said flexible backing substrate and said microneedle from said coverslip.

16. The method as recited in claim 13, wherein the lithographic fabrication of said microneedle includes direct laser writing within negative-tone photoresist applied to said surface of said coverslip.

17. A method for providing releasable tissue attachment functionality to an ingestible structure, comprising:
providing a substrate;
lithographically fabricating a microneedle unit and a displacement member on a surface of said substrate, said displacement member being secured to said microneedle unit at a first end of said displacement member, said microneedle unit including:
a microneedle defining a microneedle outer surface and a microneedle axis line extending in a longitudinal direction; and
a plurality of barb members secured to said microneedle outer surface and extending from said microneedle outer surface, said plurality of barb members being adapted for engagement with internal tissues of a subject's body;
securing a second end of said displacement member opposite said first end to an external surface of a structure body member of said ingestible device; and
applying a dissolvable coating to contain said microneedle unit and said displacement member and thereby place said displacement member in a compressed state.

18. The method of claim 17, wherein the lithographic fabrication includes direct laser writing within negative-tone photoresist applied to a surface of said substrate.

19. The method of claim 18, further comprising applying a transparent film to the surface of the substrate prior to the lithographic fabrication, wherein the direct laser writing is targeted at least partially within the transparent film.

20. The method of claim 17, wherein the dissolvable coating is applied by:
providing a perforated film having a hole formed therein;
centering said hole of said perforated film over said microneedle unit;
applying a droplet of polymer in an aqueous state to said hole of said perforated film; and
pressing said tip of said microneedle unit against said droplet until said droplet hardens.
